(19) United States
(12) Patent Application Publication
(10) Pub. No.: US 2024/0094200 A1
(43) Pub. Date: Mar. 21, 2024

(54) TARGET CAPTURE AND SENSOR ASSEMBLY FABRICATION METHOD AND TARGET CAPTURE AND SENSOR ASSEMBLY

(71) Applicant: Analog Devices, Inc., Wilmington, MA (US)

(72) Inventors: Alexander C. STANGE, Somerville, MA (US); Mohamed AZIZE, Medford, MA (US); Hari CHAUHAN, Windham, NH (US)

(73) Assignee: Analog Devices, Inc., Wilmington, MA (US)

(21) Appl. No.: 17/947,007

(22) Filed: Sep. 16, 2022

(51) Int. Cl. G01N 33/543; G01N 33/531

(52) U.S. Cl. CPC G01N 33/54353; G01N 33/531

(57) ABSTRACT

The present disclosure provides a method of fabricating a target capture and sensor assembly. The method comprises the steps of: providing a fluid path with a target capture surface comprising an anchor species with a first functional group disposed thereon; providing a target capture species to the target capture surface of the fluid path, wherein each target capture species comprises a target capture part and a second functional group configured to react with the first functional group; and exposing at least a portion of the target capture surface of the fluid path to photo radiation so as to cause a photo-initiated reaction between the first functional group and the second functional group, wherein the target capture and sensor assembly further comprises a sensing surface in the fluid path and wherein the target capture surface and the sensing surface are in fluid communication with one another.

### TECHNICAL FIELD OF THE DISCLOSURE

This disclosure relates to target capture and sensor assembly fabrication methods and target capture assemblies.

### BACKGROUND

Various filter assemblies and devices are known which filter different substances or target species such as molecules and ions. Filters work to remove these substances from a mixture based on the features of the mixture components such as particle size (for example using filters such as mesh or paper with varying pore sizes) or chemical identity (for example using an activated carbon filter in a water filtration apparatus to remove contaminants). Filters comprise filters surfaces and suffer from various drawbacks. For example, filter surfaces often lack the ability to selectively remove different types of substances from mixtures efficiently. Moreover, filter surfaces can become clogged depending on the identity of the materials being used and the frequency of the filter use. Accordingly, the efficiency of the filtration assembly/device can be significantly reduced.

The functionalisation of surfaces is known generally, for example on sensor surfaces. Functionalisation of surfaces typically involves the incorporation of chemical functionality on the surface to modify the chemical properties of said surface. For example, this could be to incorporate hydrophobic functionality through grating of polymer chains onto a gold substrate. Often, surface functionalisation is used to enhance the availability of a surface for chemical reaction.

There are various methods used to achieve this functionalisation such as drop-casting molecules in solution onto the surface. The main drawback with drop-casting is the coffee ring effect (CRE) which can occur. The CRE is the formation of a ring-like pattern on evaporation of droplets containing suspended particles onto a surface. The CRE disrupts the distribution of particles on a surface. Moreover, surfaces are commonly addressed individually with manual or even automated pipettes/nozzles. This is a serial process and may result in inefficiencies when scaling up to hundreds or thousands of functionalized areas per surface.

Accordingly, there is a need to provide an improved fabrication method for an assembly capable of selective filtration which forgoes the various challenges associated with surface functionalisation and which has the potential for efficient scale-up.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIGS. 1A and 1B provide a schematic cross-sectional view of a target capture and sensor assembly in a target capture and sensor assembly fabrication method according to an embodiment;

FIG. 2 provides a schematic cross-sectional view of a target capture and sensor assembly comprising two different target capture sites in a target capture and sensor assembly fabrication method according to an embodiment;

FIGS. 3A-3G provide a schematic plan view of a target capture and sensor assembly comprising a sensing surface disposed in the fluid path in a target capture and sensor assembly fabrication method according to an embodiment;

FIGS. 4A and 4B provide a schematic cross-sectional view of a target capture and sensor assembly comprising a sensing surface disposed in the fluid path in a target capture and sensor assembly fabrication method according to an embodiment;

FIGS. 5A and 5B provide a schematic cross-sectional view of a target capture and sensor assembly comprising a sensing surface disposed in the fluid path in a target capture and sensor assembly fabrication method according to an embodiment; and

FIG. 6 provides a block diagram of a target capture and sensor assembly fabrication method according to an embodiment.

### DESCRIPTION OF EXAMPLE EMBODIMENTS OF THE DISCLOSURE

Overview

The systems, methods and devices of this disclosure each have several innovative aspects, no single one of which is solely responsible for all of the desirable attributes disclosed herein. Details of one or more implementations of the subject matter described in this specification are set forth in the description below and the accompanying drawings.

The present disclosure provides a method of fabricating a target capture and sensor assembly in which a target capture surface has an anchor species provided thereon, the anchor species having a first functional group attached. The target capture surface is within a fluid path. The target capture species comprises a second functional group configured to react with the first functional group. The surface is then exposed to photo radiation and the first and second functional groups react forming coupling the target capture species and the anchor species on the target capture surface. The fluid path further comprises a sensing surface which is in fluid communication with the target capture surface.

In one embodiment, a method of fabricating a target capture and sensor assembly comprises the steps of: providing a fluid path, the fluid path comprising a target capture surface, wherein the target capture surface is provided with an anchor species thereon, the anchor species comprising a first functional group; providing a target capture species to the target capture surface, wherein each target capture species comprises a target capture part with an affinity for a first target and a second functional group configured to react with the first functional group; and exposing at least a portion of the target capture surface to photo radiation so as to cause a photo-initiated reaction between the first functional group and the second functional group to thereby couple the target capture species to the anchor species so as to form a target capture surface with a target capture species thereon, wherein the target capture and sensor assembly further comprises a sensing surface in the fluid path and wherein the target capture surface and the sensing surface are in fluid communication with one another.

In one embodiment, a target capture and sensor assembly is obtained or obtainable using the methods disclosed herein.

In one embodiment, a target capture and sensor assembly is provided comprising a fluid path comprising a target capture surface with a bridging species provided thereon; a target capture species coupled to the target capture surface through the bridging species, wherein the bridging species comprises a product of a photo-initiated reaction between a first functional group connected to the target capture surface via an anchor species and a second functional group connected to the target capture species; and a sensing surface, wherein the target capture surface and the sensing surface are in fluid communication with one another.

Filtration devices exist comprising filter surfaces and these also suffer from drawbacks which impact on the efficiency of the device such as lack of potential selectivity on the filter surface and clogging of filter surfaces. Functionalisation of a surface could provide a solution to a lack of selectivity. Various methods for surface functionalisation are known, for example in the context of sensors; however, these methods are inefficient owing to the lack of homogeneity of layers formed as a result of various processing effects e.g. the CRE. Accordingly, the present disclosure provides for a method of fabrication of a target capture and sensor assembly which avoids these problems.

Target Capture and Sensor Assembly Method

In one embodiment, a method of fabricating a target capture and sensor assembly comprises the steps of: providing a fluid path, the fluid path comprising a target capture surface, wherein the target capture surface is provided with an anchor species thereon, the anchor species comprising a first functional group; providing a target capture species to the target capture surface, wherein each target capture species comprises a target capture part with an affinity for a first target and a second functional group configured to react with the first functional group; and exposing at least a portion of the target capture surface to photo radiation so as to cause a photo-initiated reaction between the first functional group and the second functional group to thereby couple the target capture species to the anchor species so as to form a target capture surface with a target capture species thereon, wherein the target capture and sensor assembly further comprises a sensing surface in the fluid path and wherein the target capture surface and the sensing surface are in fluid communication with one another.

The term “target” is defined as any species which can be captured by a target capture species disposed on a target capture surface in a fluid path. Capture can include any kind of chemical interaction which is strong enough to retain the target on the target capture species. This can include covalent bond formation, as well as weaker interactions such as ionic bonds and/or van der Waals forces. Targets can be added in a mixture form to the fluid path, said mixture containing various types of mixture species. An example of a mixture may comprise target species for capture and analyte species for sensing. The targets may be captured on the target capture surface prior to the provision of the analytes to the sensing surface. The mixture can therefore be filtered of the targets prior to sensing of the analytes (i.e. post-filtration sensing of the mixture).

Embodiments therefore advantageously provide a method of fabricating a target capture device which can act as a selective filter for a particular “target” through the use of surface functionalisation and which incorporates a surface for target capture that forgoes the disadvantages associated with existing surface functionalisation methods. The target capture surface is functionalized with target capture species by reaction of the first functional group on the target capture surface with the second functional group on the target capture species through exposure to photo radiation. In particular, the reaction between the first and second functional groups using this type of “click chemistry” reaction can result in the formation of uniformly distributed monolayers which is in contrast to the uneven functionalisation distribution on the target capture surface commonly achieved through drop-casting methods (e.g. through the CRE). Accordingly, embodiments can provide for much more uniform layers of target capture species to be achieved on the target capture surface and selective capture.

Moreover, the photo-initiation of the click chemistry reaction through the exposure is fast, clean (with very few side products) and selective. The photo-initiated reaction is selective as only the functional groups on the anchor species and target capture species of complementary reactivity cause a photo click chemistry reaction to proceed so that a target capture species can be bonded to the selected target capture sites through a click chemistry bridge. The selectivity allows for those portions of the surface which were not exposed to the radiation to be subject to reaction with different target capture species (that bind different targets) in a following step upon repetition of the selective radiation exposure. Accordingly, multiple different targets can be filtered through this target capture and sensor assembly fabrication method.

Moreover, the manufacturing process and device are both more customizable, allowing for easy adaptation of the design of the target capture and sensor assembly. That is, a base structure with the anchor species in place on the target capture surface within the fluid path can be used as a framework for a single or a number of different target capture sites. Such further adaptation need not take place during fabrication, and instead could be carried out locally by a user with a specific need. For example, it might be that the target capture species is provided by an end user seeking to finalize the assembly for a particular target. In other examples, the sensing surface can be positioned anywhere along the fluid path.

In an embodiment, a target capture and sensor assembly is obtained or obtainable according to any of the above-mentioned methods.

In one embodiment, a target capture and sensor assembly comprises: a fluid path comprising a target capture surface with a bridging species provided thereon; a target capture species coupled to the target capture surface through the bridging species; wherein the bridging species comprises a product of a photo-initiated reaction between a first functional group connected to the target capture surface via an anchor species and a second functional group connected to the target capture species; and a sensing surface, wherein the target capture surface and the sensing surface are in fluid communication with one another.

Embodiments therefore advantageously provide a target capture and sensor assembly that has an improved functional layer structure. The surface has a uniformly distributed monolayers, in contrast to the uneven functionalisation distribution on surfaces using conventional surface coating techniques such as drop-casting methods (e.g. through the CRE).

Fluid Path

The method can be used to fabricate target capture and sensor assemblies which comprise fluid paths with different configurations.

The fluid path comprises the target capture surface and sensing surface. The target capture surface and the sensing surface are in fluid communication with one another. The target capture surface and sensing surface can be arranged anywhere in the fluid path so as to maintain this fluid communication. For example, in some embodiments, the target capture surface is upstream of the sensing surface. In this way, a mixture added to the fluid path may be filtered of targets at the target capture surface and then flow in the direction of the sensing surface carrying only remaining analytes to the sensing surface for detection. In some embodiments, the target capture surface is downstream of a sensing surface. In this way, a mixture comprising target molecules may be sensed prior to capture. For example, this could be useful in an application such as waste treatment whereby undesirable targets could be first be detected on the sensing surface and then filtered out of the mixture prior to release of the mixture to the environment.

In some embodiments, the fluid path comprises a fluid channel comprising the target capture surface. The fluid channel is disposed over the target capture surface so as to provide fluid to the target capture surface. Certain embodiments provide the fluid channel as a housing provided over the target capture surface. In one embodiment the fluid channel is disposed over the target capture surface. This provides the target capture surface with access to the target capture species which are added to the fluid path prior to the addition of a target. By disposing a fluid channel over the target capture surface prior to the addition of the target capture species, the chemistry of the target capture surface comprising the target capture species remains intact as it has not been exposed to harsh processing stages such as encapsulation. This allows for the target capture surface to capture targets more effectively.

In some embodiments, the assembly comprises at least one well and the target capture surface is disposed above the sensing surface in the well. This can allow for target capture prior to sensing of an analyte or sensing of the reduction of a target in a mixture as capture occurs on the target capture surface in a well environment. There may be plural wells comprising plural sensors. For example, in an embodiment, the target capture surface is disposed in a fluid path formed as part of a well. In an embodiment, the target capture surface is disposed on the sides of the well and the sensing surface is disposed within the well of the well. The well can be a microwell.

In some embodiments, the fluid channel is an enclosed fluid channel. In one embodiment, the target capture surface defines a portion of the fluid channel so that the target capture surface is enclosed within the fluid channel and is exposed to fluid passing directly through the fluid channel. By “enclosed fluid channel” it is meant that at least a portion (and in some embodiments all) of the channel is enclosed on all sides, forming a fluid conduit. This can be enclosed by the walls of the fluid channel entirely or by the wall(s) of the fluid channel being provided against another surface, e.g. the sensing surface. The method is particularly effective as it also permits the easy functionalisation of interior surfaces of an enclosed fluid channel, which other functionalisation methods cannot permit. For example, the surface may be first provided with the anchor species (this includes where the anchor species is an integral part of the channel, such as the material from which the channel is formed) and then formed. Alternatively, the channel may first be formed, followed by the securing of the anchor species followed by subsequent functionalisation with the capture species. This is advantageous as the two steps can be carried out in different environments or at different times relative to other method steps such that the functionalisation with the capture species is carried out after any potentially damaging steps.

In some embodiments, the fluid path comprises a fluid channel that is configured as a circular channel and/or comprises at least one portion that is curved; and/or the fluid path is configured as a spiral channel and/or comprises at least one spiral portion. The different fluid path configurations can provide optimized flow of substances through the fluid channel to reach the target capture surface. Moreover, the different shapes can allow for the increase in availability of target capture sites along any given fluid channel. This can improve filtering of mixtures added to the fluid path as the target capture sites can be distributed in a predetermined manner along the target capture surface.

In some embodiments, the fluid path and/or channel optionally include pump(s), valve(s) and other structures to improve the flow of reagents across the surface or away from the surface.

In one embodiment, the fluid path comprises at least one wall that is at least partially transparent to photo radiation; and the step of exposing at least a portion of the target capture surface covered by the fluid path to photo radiation comprises directing photo radiation through the at least partially transparent wall. In this way, a photo radiation source can be used externally to the target capture and sensor assembly and irradiate the target capture surface to cause the reaction between the functional groups. This can speed up fabrication, avoid the need for more complex manufacturing processes and enable the use of off-the-shelf manufacturing tools. The wall may be a part of a fluid channel, where present. It may be part of a housing or surface defining the fluid path.

In some embodiments, the fluid path and/or channel comprises an inlet configured to allow the introduction of solutions comprising target(s) into the fluid path. The fluid path may further comprise an outlet to allow for the exit of a filtered solution which has been removed of specific targets.

Target Capture Surface Materials and Configuration

The method can be used to fabricate or manufacture target capture and sensor assemblies with different target capture surface configurations. The target capture surface can be provided in the fluid path in various different configurations such that target capture species can be provided to the fluid path and access the target capture surface for attachment thereon.

In some embodiments, the target capture surface is disposed inside the fluid path. For example, where the fluid path comprises a fluid channel, the target capture surface can be located within the fluid channel. In some embodiments the target capture surface is disposed on the fluid path. The target capture surface is accordingly in direct contact with a fluid medium that is to be added to the fluid path as the fluid path defines the flow of fluid along it.

The target capture surface can be formed from a wide-ranging number of materials, provided that it allows for immobilization of an anchor species thereon. In some embodiments, the target capture surface may be formed of or comprise the anchor species.

In some embodiments, the target capture surface is a surface which is disposed within the fluid path such that the fluid path and the target capture surface are separate entities. For example, in some embodiments, the target capture surface is a layer or a coating deposited on the inside of the fluid path (e.g. an interior surface). In embodiments, this can be a layer or coating covering a part of the interior surface or can be the entire interior surface.

The target capture surface can, for example, comprise or be formed of a polymer or combination of polymer materials, such as polydimethylsiloxane (PDMS), perfluoropolyether, polymethylmethacrylate (PMMA), polystyrene (PS) or polytetrafluorethlyene (PTFE). In other embodiments, the target capture surface may comprise or be formed of carbon (graphene, graphene oxide, or nanotubes), silicon dioxide, aluminum oxide, and/or silicon.

In some embodiments, the target capture surface is defined by the fluid path such that the structure (such as a fluid channel) defining the fluid path is integrally formed with the target capture surface. The same materials can be used as listed above.

In some embodiments, the target capture surface comprises the anchor species. In other words, the inner channel is at least partly formed from a material comprising the anchor species. In one embodiment, the target capture surface comprises a polymer comprising the anchor species comprising the first functional group. In this way, the polymer provides the first functional group available for reaction with the second functional group. In some embodiments, the polymer may be formed from monomers, where each monomer comprises the anchor species. In some embodiments, the polymer is an ostemer (off stoichiometry thiol-ene) polymer. For example, in some embodiments the polymer is an ostemer polymer wherein the anchor species comprises either a thiol or alkene as a first functional group which is provided directly for reaction with the second functional group by the polymer. Example materials are, for example, set out in Carlborg et al., ‘Beyond PDMS: off-stoichiometry thiol-ene (OSTE) based soft lithography for rapid prototyping of microfluidic devices’, Lab Chip, 2011, 11, 3136, which is incorporated herein by reference. In some embodiments, the surface of the fluid path comprises a polymer made from monomers comprising an anchor species with an alkyne first functional group such that alkyne functional groups are provided directly by the polymer for reaction with the second functional group. In some embodiments, the first functional groups as part of the polymer can be azide, tetrazole, isonitrile, tetrazine, syndone, azirine, enol, epoxide, isocyanate, hydrazone or oxime groups.

In some embodiments, the target capture surface comprises at least one protrusion or raised structure, said protrusion(s) or raised structure(s) comprising the anchor species or having the anchor species provided thereon. For example, in some embodiments the target capture surface is provided with pillars or ridges extending from the target capture surface into the fluid path. These can be micrometer-sized pillars or ridges (e.g. less than 100 micrometers, such as less than 1 micrometer). This can improve sorting/filtering of the mixture comprising the targets through the increase in surface area provided for anchor species on the target capture surface. Other structural features may also be present on the target capture surface. In some embodiments, the target capture surface is provided with a graduated change in texture along the target capture surface. In some embodiments, the target capture surface is provided with a graduated height change along the target capture surface. The structure of the target capture surface can thus be configured in different ways to enhance the target capture mechanism and improve the flow of the mixture through the channel for access to the target capture surface.

Target Capture of Multiple Targets

The method can be used to fabricate target capture and sensor assemblies which capture multiple targets.

In some embodiments, the target capture surface is provided with a second anchor species provided thereon, the second anchor species comprising a third functional group, the method further comprising: providing a second target capture species to the target capture surface of the fluid path, wherein each second target capture species comprises: a second target capture part with an affinity for a second target; and a fourth functional group configured to react with the third functional group on the second anchor species; and exposing at least a portion of the target capture surface of the fluid path to photo radiation such that the third functional group on the anchor species on said portion of the target capture surface reacts with the fourth functional group on the second target capture species so as to couple the anchor species to the second target capture species such that the target capture surface of the fluid path is provided with different target capture species thereon.

The second anchor species may be disposed anywhere on the target capture surface in relation to the first anchor species. For example, in some embodiments, the first and second anchor species are disposed along the target capture surface in an alternating manner such that the different anchor species are separated by each other along the fluid path. In some embodiments, the first and second anchor species are configured in a random arrangement on the target capture surface along the fluid path or in mixed clusters on the target capture surface.

In some embodiments, the target capture surface comprises first and second target capture sites, wherein the first target capture site has the anchor species comprising the first functional group disposed thereon and the second target capture site has the second anchor species comprising the third functional group disposed thereon, wherein the third functional group can be the same as or different to the first functional group.

The first and second anchor species may therefore be arranged on separate first and second target capture sites so as to be grouped according to anchor species identity on the target capture surface. For example, in some embodiments, a fluid path can comprise a first target capture site comprising first anchor species at a first position in the fluid path and a second target capture site comprising second anchor species at a second position in the fluid path. In embodiments, the first target capture site could be located on one wall of a fluid path and the second target capture site could be located on an opposing wall of said fluid path.

In one embodiment, the first and/or third functional group on the anchor species is selected from a thiol, alkene, alkyne, azide, tetrazole, isonitrile, tetrazine, syndone, azirine, enol, epoxide, isocyanate, hydrazone or oxime group. In one embodiment, the second and/or fourth functional group on the target is selected from a thiol, alkene, alkyne, azide, tetrazole, isonitrile, tetrazine, syndone, azirine, enol, epoxide, isocyanate, hydrazone or oxime group.

Immobilization of an Anchor Species to the Target Capture Surface

Target capture surfaces can provide immobilization of anchor species through both covalent-like interactions (e.g. chemisorption of anchor species onto the surface through chemical bond formation) and non-covalent-like interactions (e.g. physisorption of anchor species onto the surface through weaker, often van der Waals, interactions) depending on the identity of the surface and the anchor species.

For example, target capture surfaces which immobilize anchor species through physisorption are negatively charged surfaces such as metal oxides. These negatively charged metal oxide surfaces can be used to immobilize positively charged species including graft polymers such as polyethylene glycol (PEG). Alternatively, common target capture surfaces which immobilize through chemisorption include gold, silver and copper. Examples of the types of anchor species which bind to these target capture surfaces include silanes (R—Si(OH)₃) and thiols (R—SH).

In one embodiment, the step of providing a target capture surface with an anchor species provided thereon comprises providing a target capture surface and adhering an anchor species to the target capture surface. Provision of the anchor species to the target capture surface can be achieved through techniques such as spin-coating, physical vapor deposition or electrophoretic deposition. Alternatively, other methods can include immersion of the target capture surface in solution.

Embodiments therefore provide a versatile array of target capture surfaces which allow functionalisation to receive target capture molecules.

Certain embodiments provide a target capture surface with first and second target capture sites. The first target capture site has the anchor species comprising the first functional group disposed thereon and the second target capture site has a second anchor species comprising a third functional group disposed thereon. The third functional group may be the same as the first functional group or of a different identity. The target capture surface can act as one target capture site or there can be multiple target capture sites on any given target capture surface. For example, where there are multiple target capture sites, there can be greater than 1, greater than 5, or greater than 10 target capture sites on any given target capture surface. The provision of more than one target capture site on the target capture surface can allow for the capture of multiple different targets through the immobilization of different target capture species with affinities for different targets on each of the target capture sites on the target capture surface.

Anchor Species

Depending on the target capture surface, different anchor species can be used (as mentioned above). In embodiments, target capture surfaces are polymers in combination with thiol-terminated anchor species. Anchor species, such as the thiol-terminated types, allow for formation of uniformly distributed self-assembled monolayers on the target capture target capture surface.

Embodiments provide anchor species comprising a first functional group. The first functional group can react, upon photo-initiation, with a second functional group on a target capture species so as to couple the target capture species to the anchor species on the target capture surface.

Certain embodiments provide anchor species with a first functional group on a first target capture site and a second anchor species with a third functional group on a second target capture site. The third functional group may be the same as the first functional group or of a different identity. This allows for multiple targets to be captured.

In one embodiment, the first and/or third functional group on the anchor species is selected from a thiol, alkene, alkyne, azide, tetrazole, isonitrile, tetrazine, syndone, azirine, enol, epoxide, isocyanate, hydrazone or oxime group.

In one embodiment, the second and/or fourth functional group on the target is selected from a thiol, alkene, alkyne, azide, tetrazole, isonitrile, tetrazine, syndone, azirine, enol, epoxide, isocyanate, hydrazone or oxime group.

Target Capture Species and Target

Target capture species comprise a target capture part and a second functional group. The second functional group is configured to react with the first functional group of the anchor species so as to couple the analyte capture species to the target capture surface via the anchor group as described above. By ‘configured to react with the first functional group’ it is meant that the second functional group is selected based on its chemical identity and ability to react with the first functional group under photo-initiated reaction conditions.

The target capture species is coupled to the anchor species directly through reaction of the first functional group on the anchor species and the second functional group on a target capture species. When formed, the target capture species is thus linked to the anchor species via a linker such as a conjugate bridge wherein the second functional group is located on a terminal end of the linker as part of the analyte capture molecule. For example, in some cases a biotin-streptavidin conjugate bridge can link the anchor species to the target capture part of the target capture species through reaction of the first and second functional groups. Those skilled in the art will appreciate may other types of links which could be formed between the anchor species and the target capture part of the target capture species.

Any suitable target capture species can be selected, according to the target which is intended to be captured by the target capture device. For example, the target capture species may comprise an antibody with specificity for a particular antigen. In such an example, the analyte may take the form of the antigen. More generally, the target capture species may, in some embodiments, comprise at least one selected from a protein, a peptide, a carbohydrate, and a nucleic acid. The protein may, for example, be an enzyme, such as an enzyme having specificity for the analyte. In other non-limiting examples, the protein is an antibody. In the latter case, the analyte may be an antigen which is selectively bound by the antibody. The target capture species may, for instance, comprise or be defined by an antigen. In this case, the target may be a species, such as an antibody, which is selectively bound by the antigenic capture species. The antigen may be or comprise, for example, a protein, a peptide, a carbohydrate, such as a polysaccharide or glycan.

In an embodiment, the target capture species comprises an aptamer. An aptamer may be defined as an oligonucleotide or peptide configured to bind the analyte. Such an aptamer may, for example, be configured to interact with, for example bind, various target types, such as small molecules, for example amino acids or amines, proteins, metal ions, and microorganisms.

The target may, for example, be selected from a molecular species, a metal ion, a virus, and a microorganism. Biomarkers, such as a cytokine or a hormone, have relevance in the context of patient monitoring, and diagnostic testing. The target may, for instance, be a hormone selected from an eicosanoid, a steroid, an amino acid, amine, peptide or protein.

In one embodiment, the target capture surface comprises first and second target capture sites; and the first target capture site has the anchor species comprising the first functional group disposed thereon; and the second target capture site has a second anchor species comprising a third functional group disposed thereon, wherein the third functional group can be the same as or different to the first functional group. In this way, plural target capture platforms are provided, which can be used for different configurations of target capture sites. For example, this can be used for multiple target capture points. Multiple target capture sites may be part of a single target capture surface, or may be separate target capture surfaces.

Certain embodiments provide for the provision of a second target capture species to the fluid path. In particular, in an embodiment, the method comprises providing a second target capture species to the fluid path, wherein each second target capture species comprises: a second target capture part with an affinity for a different analyte; and a fourth functional group configured to react with the third functional group on the second anchor species; and exposing at least a portion of the target capture surface to photo radiation such that the third functional group on the anchor species on said portion of the target capture surface reacts with the fourth functional group on the second analyte capture species so as to couple the anchor species to the second analyte capture species such that a target capture and sensor assembly with more than one target capture surface is formed, each target capture surface with a different target capture species.

The fourth functional group on the second target capture species may be the same as or different to the second functional group on the first target capture species. Accordingly, multiple target capture sites can be provided, each with a different target capture species with an affinity for a different target provided thereon. This allows for the capture of multiple types of target to occur. The target capture surface can act as one target capture site or there can be multiple target capture sites on any given target capture surface. For example, where there are multiple target capture sites, there can be greater than 1, greater than 5, or greater than 10 target capture sites on any given target capture surface. The method is particularly advantageous in this respect since different sites can be selectively functionalized using the improved method. Moreover, the process is very straightforward compared to existing fabrication techniques, since the different surfaces can be functionalized simply by directing the light to the site of interest (e.g. using directed light sources or masks). In this way, one capture species can be provided to the fluid path (which can be in contact with both sensing sites) and only one target capture site (e.g. a portion of a target capture surface or a separate target capture surface) irradiated so as to adhere that capture species to the irradiated target capture site. Subsequently, an additional, different capture species can be provided to the fluid path and another target capture site irradiated to adhere the different capture species to the other inner site.

Reaction Between Functional Groups on Anchor Species and Target Capture Species

The reaction of the first or third functional group and the second or fourth functional group, respectively, proceeds via a click chemistry type of reaction in the presence of photo radiation. The reactions are fast and efficient (with no/few side products). Moreover, this provides a customizable fabrication platform, since there are a wide range of options available for the identity of the click chemistry functional groups on both the anchor species and the target capture species that can undergo the photo-initiated reaction. The identities of the groups on either the anchor species or the target capture species can be interchanged. The click chemistry reactive groups can therefore in some embodiments comprise or consist of, but are not limited to, combinations of the following: azide plus alkyne; thiol plus alkene; tetrazole plus alkene; and Diels Alder cis diene plus alkene reagents.

For example, the first or third functional group (FG1/FG3) can be a thiol, alkene, alkyne, azide, tetrazole, isonitrile, tetrazine, syndone, azirine, enol, epoxide, isocyanate, hydrazone or oxime group. The first or third functional group reacts with a second functional group on the analyte capture species upon photo-initiation. The second or fourth functional group (FG2/FG4) accordingly has complementary reactivity to the first of third functional group and can be any group selected from the same list for which a reaction can occur. For example some examples of the first or third and second or fourth functional group combinations can include: FG1/FG3=thiol and FG2/FG4=alkene; FG1/FG3=azide and FG2/FG4=alkyne; and FG1/FG3=tetrazole and FG2/FG4=alkene.

The photo radiation used to initiate the reaction between the first and second or third or fourth functional groups can be any suitable wavelength. In some embodiments, the method uses UV light as a photo radiation. In some embodiments, the wavelength of photo radiation can range from anywhere between from 300 to 900 nm. For example, the wavelength of photo radiation can be from 320 to 350 nm, from 355 to 370 nm, or from 600 to 900 nm. The wavelength of light photo radiation be, for example, 301 nm, 302 nm, 303 nm, 304 nm, 305 nm, 395 nm or 400 nm. A source can be selected from any photo-radiation source including, but not limited to, a scanning laser beam or LED array. The photo radiation source can be positioned in such a way to form a particular exposure pattern on the target capture surface. For example, this can be achieved by use of a photomask, projection lithography or using an array of addressable LEDs. Different types of radiation source can provide for different wavelengths of light depending upon the wavelength required for the selected first and second functional groups. In one embodiment, the step of exposing at least a portion of the target capture surface to photo radiation comprises at least one of: positioning a photomask to direct photo radiation to a specific part of the target capture surface; using projection lithography; using a scanning laser; or using an array of addressable LEDs.

In some embodiments, heat sources are introduced along the fluid path to speed up the photo-initiated reaction between first and second functional groups. Without limitation, this heating could be achieved by heat pumps dispersed throughout the fluid path or external heat sources surrounding the fluid path.

Sensing Surface

The sensing surface in the method is any surface that may be configured to sense a property of a fluid, such as a parameter (e.g. conductivity, temperature) or presence of an analyte (e.g. concentration). For example, the sensing surface may be configured to sense specific analytes, concentration of a target molecule, pressure or temperature. The sensing surface may be an electrode surface. In some embodiments, this may comprise or be formed from copper, nickel, platinum, silver, silver chloride, gold or other noble metals. In some embodiments, this may comprise or be formed from TiO₂ or indium tin oxide (ITO). Other sensing surfaces may include a substrate with a coating on which the anchor species is immobilized. For example, the sensing surface may be a glass substrate with an ITO coating thereon. In other embodiments, the sensing surface may comprise or be formed of carbon (graphene, graphene oxide, or nanotubes), silicon dioxide, aluminum oxide, and/or silicon.

Embodiments provide the sensing surface and target capture surface in fluid communication with one another. For example, in some embodiments, the sensing surface is downstream of the target capture surface in the fluid path. In this way, a mixture comprising target species and analyte species added to the fluid path can be filtered of target species in the mixture through target capture of target species on the target capture surface and the filtered solution comprising analyte species can subsequently be sensed on the sensing surface. This allows for performance of efficient sensing of a mixture following filtration of said mixture through target capture. Accordingly, the target capture species on the target capture surface allows for effective filtration of mixtures containing different targets and can also be incorporated into sensing applications. The sensing of the filtered mixture is extremely efficient as there are minimal impurities in the mixture which could cause interference to the sensing mechanism.

In some embodiments, prior to the step of providing a target capture species to the target capture surface, the method further comprises providing the anchor species to the target capture surface.

In some embodiments, the sensing surface is provided to the assembly prior to the step of providing the anchor species. In other embodiments, the sensing surface is provided to the assembly after the step of providing the target capture surface with the anchor species, but prior to the step of providing the target capture species to the target capture surface. In other embodiments, the sensing surface is provided after the provision of the target capture species to the target capture surface.

Embodiments therefore advantageously provide customisability in the fabrication of the target capture and sensor assembly as the sensing surface can be added at different points in the fabrication. By adding the sensing surface prior to the addition of the target capture species, both the target capture surface and the sensing surface can be encapsulated into a device, leaving the target capture species to be added later and thus free from exposure to harsh processing conditions used in device fabrication.

In some embodiments, the fluid path comprises a fluid channel comprising the target capture surface. In some embodiments the fluid channel further comprises the sensing surface. In some embodiments where the fluid path comprises a fluid channel, the sensing surface is external to the fluid channel.

In some embodiments, the sensing surface is functionalised with an anchor species provided thereon. This can be the same or a different anchor species to the anchor species of the target capture surface. Embodiments of anchor species set out in respect of the target capture surface apply equally to the anchor species of the sensing surface. In some embodiments, the sensing surface comprises an anchor species comprising a fifth functional group. In some embodiments, the method comprises the steps of providing an analyte capture species to the sensing surface, wherein each analyte capture species comprises an analyte capture part and a sixth functional group configured to react with the fifth functional group; and exposing at least a portion of the sensing surface to photo radiation so as to cause a photo-initiated reaction between the fifth functional group and the sixth functional group to thereby couple the analyte capture species to the anchor species on the sensing surface and form a sensing surface with an analyte capture species thereon. The functional groups can be the same or a different functional group to the functional group(s) of the target capture surface. Embodiments of functional group(s) set out in respect of the target capture surface (i.e. first to fourth) apply equally to the functional groups (i.e. fifth to eighth) of the sensing surface.

Embodiments therefore advantageously allow the chemistry of the sensing surface to remain intact prior to the addition of the analyte capture species. Furthermore, the efficiency of the method is further improved as provision of the photo radiation to the sensing surface can be done at the same time as exposure of the target capture surface to photo radiation.

Incorporation of a sensing surface comprising a fifth functional group on an anchor species improves the potential for efficient sensing (following addition of an analyte capture species to the fluid path). This is due to the improved analyte capture mechanisms that can be employed via the immobilisation of the analyte on the sensing surface via the anchor species. Accordingly, filtration of the mixture can be achieved through target capture which subsequently allows for efficient sensing of a filtered mixture, improved by the functionalisation of the sensing surface.

“Analyte” as used herein can be the same as the “target” set out herein. The term “target” is used to denote that these not necessarily be analysed, although in some embodiments the target may be an analyte. As a result, the target capture species referred to herein is defined in the same way as the analyte capture species set out herein and may have any of the features defined in respect of the target capture species. Embodiments set out with respect to either apply equally to the other. In some embodiments, the “analyte” may be the same species as the “target”. This can be advantageous in embodiments where the target capture surface is located downstream of the sensing surface. For example, this may be used to capture and retain the analyte after sensing (e.g. for disposal). In other embodiments, the “analyte” may be a different species to the “target”. In this way, the “target” may be filtered out so that a better analyte reading may be taken (e.g. if the target capture surface is upstream of the sensing surface) or for disposal of the target (whether upstream or downstream).

The reaction of the fifth functional group on the anchor species and the sixth functional group on the analyte capture species, respectively, proceeds via a click chemistry type of reaction in the presence of photo radiation. The reactions are fast and efficient (with no/few side products). Moreover, this provides a customisable fabrication platform, since there are a wide range of options available for the identity of the click chemistry functional groups on both the anchor species and the analyte capture species that can undergo the photo-initiated reaction. The identities of the groups on either the anchor species or the analyte capture species can be interchanged. The click chemistry reactive groups can therefore in some embodiments comprise or consist of, but are not limited to, combinations of the following: azide plus alkyne; thiol plus alkene; tetrazole plus alkene; and Diels Alder cis diene plus alkene reagents.

In one embodiment, the sensing surface comprises first and second sensing sites; and the first sensing site has the anchor species comprising the fifth functional group disposed thereon; and the second sensing site has a second anchor species comprising a seventh functional group disposed thereon, wherein the seventh functional group can be the same as or different to the first functional group. In this way, plural sensing platforms are provided, which can be used for different configurations of sensing sites. For example, this can be used for multiple sensing points or multiplexing. Multiple sensing sites may be part of a single sensing surface, or may be separate sensing surfaces. Each sensing surface may generate a separate signal, which can be used to infer binding on the sensing surface.

The anchor species and the second anchor species may be the same as the anchor species provided on the target capture surface. Alternatively, these may be different. Similarly, the anchor species and the second anchor species provided on the sensing surface may be the same as one another or different. In each of these embodiments, the embodiments set out in respect of the anchor species of the sensing surface apply equally to the anchor species of the target capture surface.

For example, the fifth group (FG5) and seventh group (FG7) can be a thiol, alkene, alkyne, azide, tetrazole, isonitrile, tetrazine, syndone, azirine, enol, epoxide, isocyanate, hydrazone or oxime group. The fifth functional group reacts with a sixth functional group on the analyte capture species upon photo-initiation. The sixth functional group (FG6) or eighth (FG8) accordingly has complementary reactivity to the fifth functional group and can be any group selected from the same list for which a reaction can occur. For example some examples of the fifth and sixth functional group combinations can include: FG5/FG7=thiol and FG6/FG8=alkene; FG5/FG7=azide and FG6/FG8=alkyne; and FG5/FG7=tetrazole and FG6/FG8=alkene.

The photo radiation used to initiate the reaction between the fifth and sixth functional groups can be any suitable wavelength. In some embodiments, the method uses UV light as a photo radiation. In some embodiments, the wavelength of photo radiation can range from anywhere between from 300 to 900 nm. For example, the wavelength of photo radiation can be from 320 to 350 nm, from 355 to 370 nm, or from 600 to 900 nm. The wavelength of light photo radiation be, for example, 301 nm, 302 nm, 303 nm, 304 nm, 305 nm, 395 nm or 400 nm. A source can be selected from any photo-radiation source including, but not limited to, a scanning laser beam or LED array. The photo radiation source can be positioned in such a way to form a particular exposure pattern on the target capture surface. For example, this can be achieved by use of a photomask, projection lithography or using an array of addressable LEDs. Different types of radiation source can provide for different wavelengths of light depending upon the wavelength required for the selected first and second functional groups. In one embodiment, the step of exposing at least a portion of the target capture surface to photo radiation comprises at least one of: positioning a photomask to direct photo radiation to a specific part of the target capture surface; using projection lithography; using a scanning laser; or using an array of addressable LEDs.

Target Capture and Sensor Assembly

Target capture and sensor assemblies can be provided according to any of the above-mentioned methods for target capture and sensor assembly fabrication.

In one embodiment, a target capture and sensor assembly comprises: a fluid path comprising a target capture surface with a bridging species provided thereon; a target capture species coupled to the target capture surface through the bridging species; wherein the bridging species comprises a product of a photo-initiated reaction between a first functional group connected to the target capture surface via an anchor species and a second functional group connected to the target capture species; and a sensing surface, wherein the target capture surface and the sensing surface are in fluid communication with one another.

The “bridging species” is defined as the product of reaction between the functional group on the anchor species and the functional group on the target capture species following a photo-initiated reaction. For example, this could be the product of a photo-initiated reaction between a thiol and an alkene which includes a bond formed between the sulphur of the thiol and the vinylic carbon of the alkene (in such a thiol-alkene example). Other examples of bridging species can include, but are not limited to, various bonds formed between different complementary functional groups on the anchor species and target capture species (e.g. the bond formed between the alkyne carbons on an alkyne functional group and the nitrogens on an azide functional group). In this way the bridging species can be seen as a tether on the target capture surface.

In an embodiment, a target capture and sensor assembly comprises a target capture surface with first and second target capture sites (in some embodiments, plural target capture sites), each target capture site having a bridging species disposed thereon. In some embodiments, the first target capture site has a first target capture species disposed thereon and the second target capture site has a second target capture species disposed thereon. In other words, each target capture site is provided with a different target capture species linked to the anchor species via the bridging species. Each target capture species on any target capture site can have an affinity for a different target so as to allow target capture of different targets in the same target capture and sensor assembly—i.e. the target capture and sensor assembly can allow for efficient capture of multiple targets. The target capture surface can act as one target capture site or there can be multiple target capture sites on any given target capture surface. For example, where there are multiple target capture sites, there can be greater than 1, greater than 5, or greater than 10 target capture sites on any given target capture surface. For example, there can be 2, 3, 4, 5, 6, 7, 8, 9, 10, 20 or 100 target capture sites.

In some embodiments, the sensing surface comprises; a further bridging species provided thereon; and an analyte capture species coupled to the sensing surface through the further bridging species; wherein the further bridging species comprises a product of a photo-initiated reaction between a fifth functional group connected to the sensing surface via an anchor species and a sixth functional group connected to the analyte capture species.

### Example Embodiments

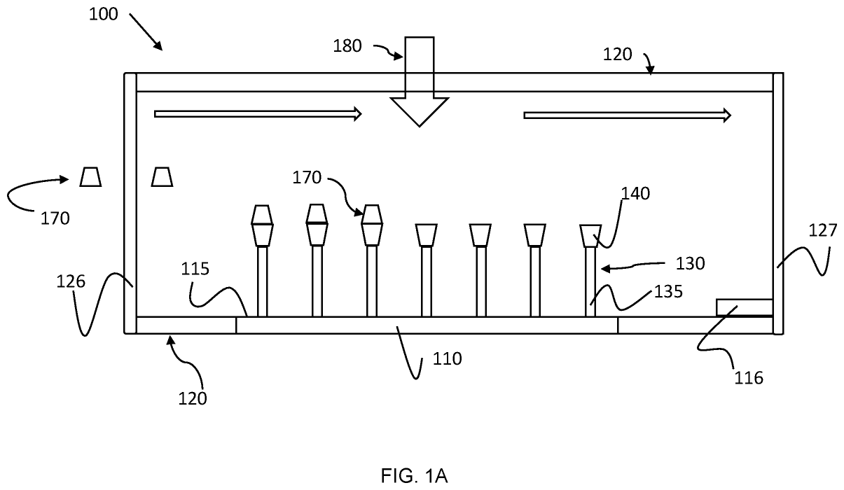

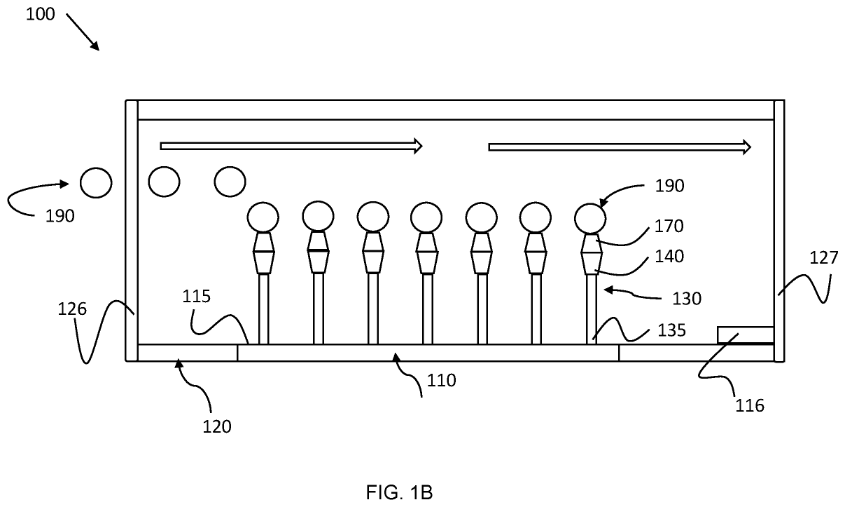

A first embodiment of a method for target capture and sensor assembly fabrication 100 is shown is FIGS. 1A and 18. FIG. 1A shows a fluid path 120 is provided as a fluid channel with an inlet 126 and on outlet 127. The fluid path 120 comprises a target capture surface 110 with an upper face 115 and a sensing surface 116 disposed on the fluid path downstream of the target capture surface. The target capture surface 110 is defined by a part of the fluid path 120. A fluid medium can be provided to the target capture surface 110 directly through the inlet 126. The arrows show the direction of fluid flow through the fluid path 120.

The target capture surface 110 is provided with anchor species 130 adhered on the upper face 115, with each anchor species 130 comprising a first functional group 140 and a tail portion 135 which immobilizes the anchor species 130 onto the upper face 115 of the target capture surface 110. The method comprises providing a fluid comprising a target capture species which has a second functional group 170 capable of reaction with the first functional group 140 on the anchor species 130. The target capture surface 110 is irradiated with photo radiation 180 in the presence of the target capture species 170. Upon exposure of the surface to the photo radiation, the first functional group 140 reacts with the second functional group 170 so as to couple the target capture species 170 to the surface 110. This forms a fluid path 120 which includes a target capture species part 170 adhered thereto.

The use of the fluid path 120 with the capture species part 170 adhered thereto is depicted in FIG. 18. Here, a target 190 is provided to the fluid path 120 via the inlet 126 and flowed over the target capture surface 110. The target 190 adheres to the capture species part 170 on the target capture surface 110 thereby adhering the target 190 to the target capture surface 110. In this way, target 190 is filtered out of the fluid that is flowed through the fluid path 120. The filtered fluid can then be received by the sensing surface 116.

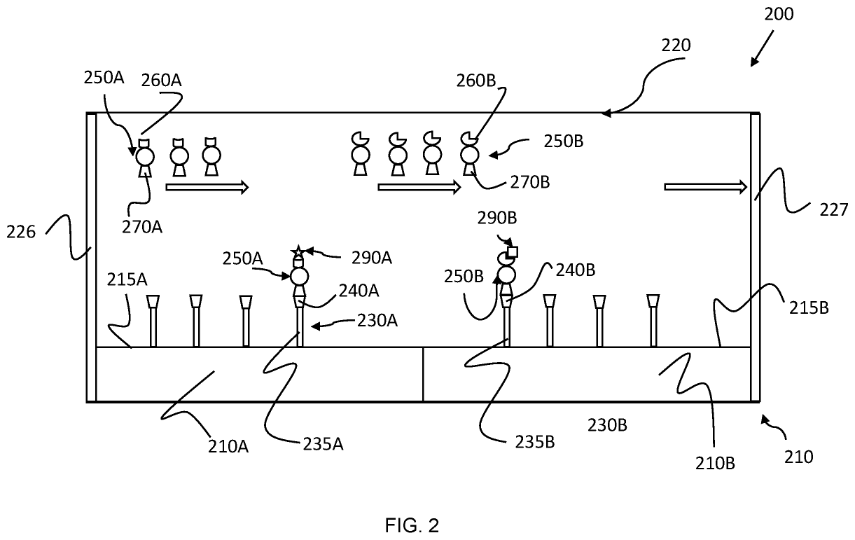

Another embodiment of the method of fabricating a target capture and sensor assembly 200 is shown in FIG. 2. FIG. 2 shows a fluid path 220 comprising a target capture surface 210 which is an elongate strip comprising two target capture sites 210A and 210B as two connected portions which make up the whole target capture surface 210. Each target capture site has a respective upper face portion 215A, 215B. Upper face portion 215A has a first anchor species 230A disposed thereon and upper surface portion 215B has a second anchor species 230B disposed thereon. Each anchor species 230A, 230B comprises a respective tail portion 235A, 235B and a functional group. Anchor species 230A comprises a first functional group 240A configured to react with a second functional group 270A of a first target capture species 250A and anchor species 230B comprises a third functional group 240B configured to react with a fourth functional group 270B of a second target capture species 250B. Each anchor species 230A, 230B is immobilized on the respective upper face portion 215A, 215B of the target capture site through a respective tail portion 235A, 235B. FIG. 2 further shows the target capture surface is defined by the fluid path 220. The fluid path 220 comprises an inlet 226 for addition of reagents into the fluid path 220 and outlet 227. The fluid path 220 further comprises a sensing surface however this is not shown. A first target capture species 250A is added to the fluid path 220 through inlet 226, in addition to a second target capture species 250B. The second target capture species 250B has an affinity for a different target 290B compared to the first target capture species 250A with affinity for target 290A. First target capture site 210A is exposed to photo radiation separately to second target capture site 210B. Accordingly, on exposure to the photo radiation, the first functional group 240A on the anchor species 230A reacts with the second functional group 270A on the first target capture species 250A. Separately, on exposure to photo radiation, the third functional group 240B on the anchor species 230B reacts with the fourth functional group 270B on the second target capture species 250B. Accordingly, the target capture surface 110 comprises first target capture site 210A coupled to first target capture species 250A which has an affinity for target 290A and second target capture site 210B is coupled to second target capture species 250B which has an affinity for target 290B. Multiple target capture sites can be provided; however, only one further target capture site is shown for ease of illustration. Multiple further target capture species can therefore be added to the fluid path however only one further target capture species is shown for ease of illustration. Each further target capture species has an affinity for a different target.

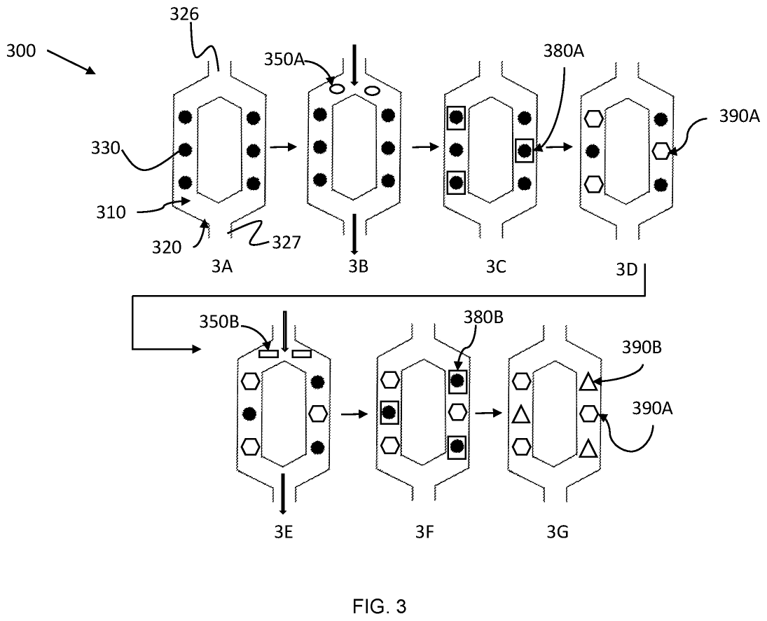

A further embodiment of the method of fabricating a target capture and sensor assembly 300 is shown in FIGS. 3A-3G. FIG. 3A shows a target capture surface 310 disposed within a fluid path 320. The fluid path 320 is configured as two pathways which branch from an initial inlet 326 and which lead to an outlet 327 where the two pathways recombine. The target capture surface 310 comprises anchor species 330. The target capture surface 310 is disposed along each pathway so that anchor species 330 are disposed on each pathway. As for the previous embodiments, each anchor species 330 comprises a tail portion and a first functional group (not shown) and the anchor species 330 is immobilized on the target capture surface 310 through the tail portion. FIG. 3B shows target capture species 350A added to fluid path 320. As for previous embodiments, target capture species 350A comprises a target capture part and a second functional group configured to react with the first functional group of the anchor species (not shown). FIG. 3C shows, in a next step, the anchor species 330 is selectively exposed to photo radiation through a photomask to form selected anchor species 380A with a target capture species 350A bound thereto. Whilst not shown, the photomask is directly above the fluid path 310 and is configured to mask sites which are not intended to bind a target capture species 350A. FIG. 3D shows that, in a next step, a target is added to the fluid path and binds to the target capture species 350A to form an anchor species with a target bound thereto 390A via the target capture species. Accordingly, the target capture surface 310 comprises anchor species 330 which remains unbound and anchor species with a target bound thereto 390A. FIG. 3E shows the continuation of the method of fabrication with the addition of a different target capture species 350B to the fluid path 320. FIG. 3F shows that the remaining unbound anchor species 330 are selectively exposed to photo radiation through a photomask to from selected anchor species 380B with a target capture species 350B bound thereto. FIG. 3G shows, in a next step, a different analyte with an affinity for target capture species 350B is added to the fluid path 320 to form an anchor species with a target bound thereto 390B via the target capture species. Accordingly, FIG. 3G shows the target capture surface 310 comprising two different types of target bound to anchor species, 390A and 390B. Multiple further target capture species can be added to the fluid path however only one further target capture species is shown for ease of illustration. Each further target capture species has an affinity for a different target. Whilst not shown, the fluid path 320 further comprises a sensing surface disposed along it downstream of outlet 327.

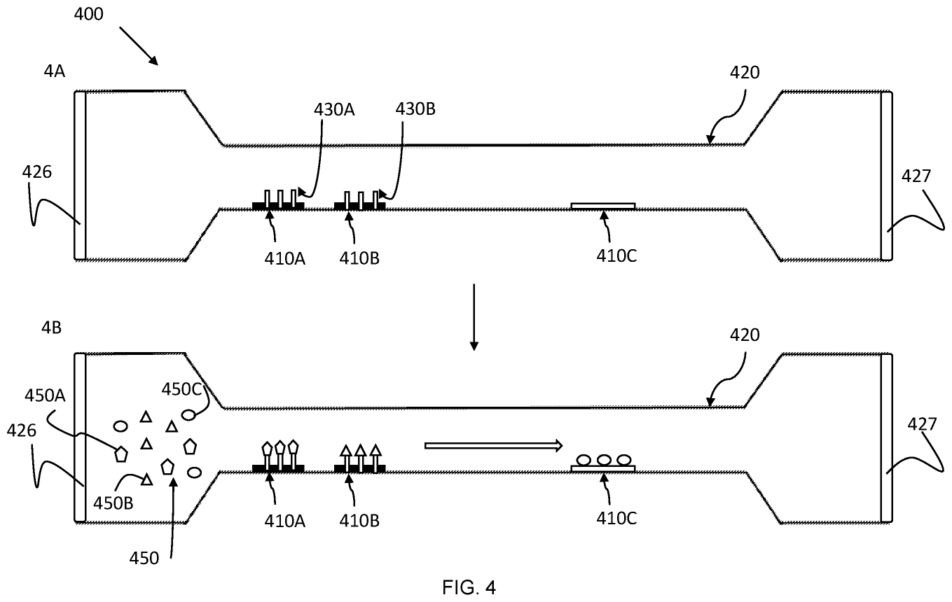

A further embodiment of the method of fabricating a target capture and sensor assembly 400 is shown in FIGS. 4A-4B. FIG. 4A shows a fluid path 420 provided as a fluid channel with an inlet 426 and outlet 427. The fluid path comprises target capture surfaces 410A and 410B. The target capture surfaces 410A and 410B are respectively provided with target capture species 430A and 430B bound thereto in the same way as described with reference to the previous embodiment shown in FIG. 1A (i.e. through reaction of a first functional group on an anchor species with a second functional group on a target capture species in the presence of photo radiation, not shown). The fluid path 420 further comprises a sensing surface 410C. The sensing surface 410C can be any sensing surface capable of analyte or variable detection and is provided along the fluid path 420 after the target capture surfaces 410A and 410B. FIG. 4B shows the addition of a mixture 450 comprising targets 450A and 450B in addition to analytes 450C to the fluid path 420. The fluid flows through the fluid path 420 as indicated by the direction of the arrow within the fluid path 420. As the mixture 450 flows through the fluid path 420, the targets 450A and 450B are captured by the respective target capture species 430A and 430B bound to the respective surfaces 410A and 410B. The remaining analytes 450C in the mixture 450 are then able to flow over the sensing surface 410C for detection. Accordingly, the mixture 450 is filtered leaving the analytes 450C for detection over the sensing surface 410C.

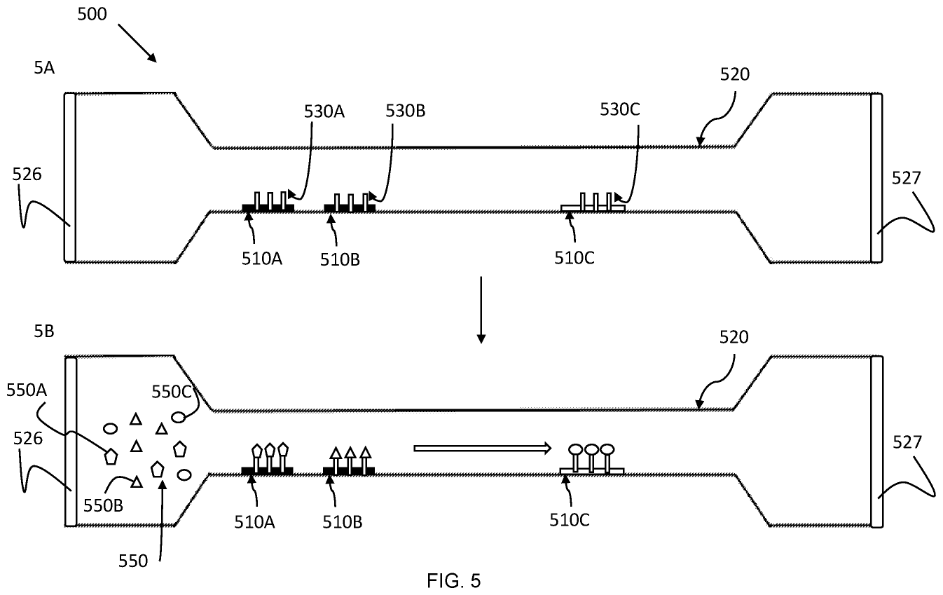

A further embodiment of the method of fabricating a target capture and sensor assembly 500 is shown in FIGS. 5A-5B. A fluid path 520 is provided as configured in the previous embodiment with target capture surfaces 510A and 510B, inlet 526 and outlet 527. The target capture surfaces 510A and 510B are respectively provided with target capture species 530A and 530B bound thereto in the same way as described with reference to the previous embodiments and as shown in FIG. 1A (i.e. through reaction of a first functional group on an anchor species with a second functional group on a target capture species in the presence of photo radiation). The fluid path 520 further comprises a sensing surface 510C. The sensing surface 510C comprises an analyte capture species bound to an anchor species 530C disposed on the sensing surface 510C. The sensing surface 510C is disposed within the fluid path along the path of fluid flow after the target capture surfaces 510A and 510B. FIG. 5B shows the addition of a mixture 550 comprising targets 550A and 550B in addition to analytes 550C to the fluid path 520. The fluid flows through the fluid path 520 as indicated by the direction of the arrow within the fluid path 520. As the mixture 550 flows through the fluid path 520, the targets 550A and 550B are captured by the respective target capture species 530A and 530B bound to the respective surfaces 510A and 510B. The remaining analytes 550C in the mixture 550 are then able to flow over the sensing surface 510C for detection. Accordingly, the mixture 550 is filtered leaving the analytes 550C for detection over the sensing surface 510C.

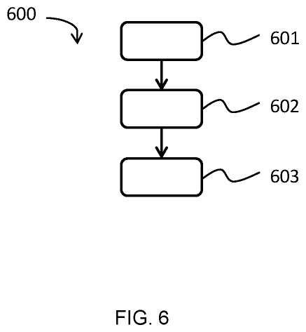

FIG. 6 provides a block diagram of a method of target capture and sensor assembly fabrication 600 according to an example. The step of providing a fluid path comprising a target capture surface, wherein the target capture surface is provided with an anchor species thereon, the anchor species comprising a first functional group is represented by 601; providing a target capture species to the target capture surface of the fluid path, wherein each target capture species comprises a target capture part with an affinity for a first target and a second functional group configured to react with the first functional group is represented by 602; and exposing at least a portion of the target capture surface to photo radiation so as to cause a photo-initiated reaction between the first functional group and the second functional group to thereby couple the target capture species to the anchor species so as to form a target capture surface with a target capture species thereon, wherein the target capture and sensor assembly further comprises a sensing surface in the fluid path and wherein the target capture surface and the sensing surface are in fluid communication with one another is represented by 603.

### EXAMPLES

Examples of specific capture species and functional groups will now be set out.

In an example, a target capture and sensor assembly is provided comprising a fluid path, at least a part of which defines a surface made out of ostemer polymer. The ostemer polymer comprises excess thiol groups on the surface. A medium comprising a target capture species is added to the fluid path. The fluid path is exposed to UV photo radiation and target capture species with a complementary functional group to the thiol group are subsequently bonded to the thiol groups. For example, target capture species with alkene functionalisation are added and are bonded to the thiol groups on the surface using photo radiation and a photo ‘click’ reaction.

In an example, a method of fabricating a target capture and sensor assembly comprises the step of providing a fluid path with a sensing surface. The assembly comprises a fluid path which comprises a target capture surface with an anchor species disposed thereon. The anchor species comprises thiol first functional groups disposed thereon. A mixture comprising a target capture species is added to the fluid path. The fluid path is exposed to UV photo radiation and an alkene-terminated target capture species is subsequently bonded to the thiol group. The fluid path further comprises a sensing surface. The sensing surface can be made out of any material capable of analyte detection, e.g. a gold electrode. Following the binding of the target capture species to the target capture surface, a mixture comprising targets and analytes A is added to the fluid path. The mixture flows over the target capture surface first and the targets are subsequently bound to the target capture species on the target capture surface. The mixture is accordingly filtered of the targets and the analytes A are free to be detected on the sensing surface.

In another example, a method of fabricating a target capture and sensor assembly is provided in accordance with the previous example; however, the sensing surface is a SiO₂ surface comprising thiol-terminated anchor species. Alkene-terminated biotinylated analyte capture species are added to the fluid path following the step of binding the target capture species to the anchor species on the target capture surface. The fluid path is then selectively exposed to UV photo radiation so as to bind the analyte capture species to the sensing surface via the anchor species. A mixture comprising targets and analytes A is then added to the fluid path. The mixture flows over the target capture surface first and the targets are subsequently bound to the target capture species on the target capture surface. The mixture is accordingly filtered of the targets and the analytes A then bind to the analyte capture species on the sensing surface for detection.

In any of the above examples, the alkene-terminated biotinylated species may, for example be an aptamer or an antibody and the analytes A and B may be selected from COVID-19, MERS, Influenza A or Influenza B. For example, an alkene-terminated biotinylated aptamer can be used which is specific to any of COVID-19, MERS, Influenza A or Influenza B. Likewise, an alkene-terminated biotinylated antibody may be used which is specific to any of COVID-19, MERS, Influenza A or Influenza B.

Example materials of the sensing surface and specific alkene-terminated biotinylated species are, for example, set out in Waldmann et al., ‘Preparation of Biomolecule Microstructures and Microarrays by Thiol-ene Photoimmobilization’, ChemBioChem, 2010, 11, 235, which is incorporated herein by reference.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present disclosure can be better understood from the description, appended claims or aspects, and accompanying drawings. It should be understood that the drawings are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

Numerous other changes, substitutions, variations, alterations, and modifications may be ascertained to one skilled in the art and it is intended that the present disclosure encompass all such changes, substitutions, variations, alterations, and modifications as falling within the scope of the appended select examples. Note that all optional features of the apparatus described above may also be implemented with respect to the method or process described herein and specifics in the examples may be used anywhere in one or more embodiments.

1. A method of fabricating a target capture and sensor assembly, the method comprising:
providing a fluid path, the fluid path comprising a target capture surface, wherein the target capture surface is provided with an anchor species thereon, the anchor species comprising a first functional group;
providing a target capture species to the target capture surface, wherein each target capture species comprises a target capture part with an affinity for a first target and further comprises a second functional group configured to react with the first functional group; and
exposing at least a portion of the target capture surface to photo radiation to cause a photo-initiated reaction between the first functional group and the second functional group to couple the target capture species to the anchor species so as to form a target capture surface with a target capture species thereon,
wherein the target capture and sensor assembly further comprises a sensing surface in the fluid path and wherein the target capture surface and the sensing surface are in fluid communication with one another.

2. The method according to claim 1, wherein, prior to providing a target capture species to the target capture surface, the method includes providing the target capture surface with the anchor species thereon.

3. The method according to claim 2, wherein the sensing surface is provided prior to providing the target capture surface the anchor species.

4. The method according to claim 2, wherein the sensing surface is provided after providing the target capture surface with the anchor species, but prior to providing the target capture species to the target capture surface.

5. The method according to claim 1, wherein the fluid path comprises a fluid channel and wherein the fluid channel comprises the target capture surface.

6. The method according to claim 5, wherein at least one of:
the fluid channel is a circular channel,
the fluid channel comprises at least one portion which is curved,
the fluid channel is a spiral channel, or
the fluid channel comprises at least one spiral portion.

7. The method according to claim 1, wherein the target capture surface is provided with a second anchor species thereon, the second anchor species comprising a third functional group, and wherein the method further comprises:
providing a second target capture species to the target capture surface, wherein each second target capture species comprises a second target capture part with an affinity for a second target and further comprises a fourth functional group configured to react with the third functional group on the second anchor species; and
exposing at least a portion of the target capture surface to photo radiation to cause the third functional group on the anchor species on said portion of the target capture surface to react with the fourth functional group on the second target capture species so as to couple the anchor species to the second target capture species such that the target capture surface is provided with different target capture species thereon.

8. The method according to claim 7, wherein:
the target capture surface comprises first and second target capture sites;
the first target capture site has the anchor species comprising the first functional group disposed thereon; and
the second target capture site has the second anchor species comprising the third functional group disposed thereon, wherein the third functional group can be the same as or different to the first functional group.

9. The method according to claim 7, wherein:
at least one of the first functional group on the anchor species and the third functional group on the anchor species includes at least one of a thiol, alkene, alkyne, azide, tetrazole, isonitrile, tetrazine, syndone, azirine, enol, epoxide, isocyanate, hydrazone or oxime group, or
at least one of the second functional group on the target and the fourth functional group on the target includes at least one of a thiol, alkene, alkyne, azide, tetrazole, isonitrile, tetrazine, syndone, azirine, enol, epoxide, isocyanate, hydrazone or oxime group.

10. The method according to claim 1, wherein the target capture surface comprises the anchor species.

11. The method according to claim 1, wherein the target capture surface comprises a polymer comprising the anchor species.

12. The method according to claim 11, wherein the polymer comprising the anchor species is an off stoichiometry thiol-ene polymer.

13. The method according to claim 1, wherein:
the fluid path comprises at least one wall that is at least partially transparent to photo radiation, and
exposing at least a portion of the target capture surface to photo radiation comprises directing photo radiation through the at least partially transparent wall.

14. The method according to claim 1, wherein the target capture surface includes raised structures, the raised structures comprising anchor species thereon.

15. The method according to claim 1, wherein exposing at least a portion of the target capture surface to photo radiation comprises at least one of:
positioning a photomask to direct photo radiation to a specific part of the target capture surface,
projecting a pattern onto the surface using projection lithography,
using a scanning laser,
or using an array of addressable light-emitting diodes.

16. The method according to claim 1, wherein the sensing surface comprises an anchor species comprising a fifth functional group.

17. The method according to claim 16, further comprising:
providing an analyte capture species to the sensing surface, wherein each analyte capture species comprises an analyte capture part and a sixth functional group configured to react with the fifth functional group; and
exposing at least a portion of the sensing surface to photo radiation so as to cause a photo-initiated reaction between the fifth functional group and the sixth functional group to thereby couple the analyte capture species to the anchor species on the sensing surface and form a sensing surface with an analyte capture species thereon.

18. A target capture and sensor assembly comprising:
means for providing a fluid path comprising a target capture surface, wherein the target capture surface includes an anchor species thereon, the anchor species comprising a first functional group;
means for providing a target capture species to the target capture surface, wherein each target capture species comprises a target capture part with an affinity for a first target and further comprises a second functional group configured to react with the first functional group; and
means for exposing at least a portion of the target capture surface to photo radiation to cause a photo-initiated reaction between the first functional group and the second functional group to couple the target capture species to the anchor species so as to form a target capture surface with a target capture species thereon,
wherein the target capture and sensor assembly further comprises a sensing surface in the fluid path and wherein the target capture surface and the sensing surface are in fluid communication with one another.

19. A target capture and sensor assembly comprising:
a fluid path comprising a target capture surface with a bridging species thereon;
a target capture species coupled to the target capture surface through the bridging species, wherein the bridging species comprises a product of a photo-initiated reaction between a first functional group connected to the target capture surface via an anchor species and a second functional group connected to the target capture species; and
a sensing surface, wherein the target capture surface and the sensing surface are in fluid communication with one another.

20. The target capture and sensor assembly according to claim 19, wherein the sensing surface comprises:
a further bridging species provided thereon; and
an analyte capture species coupled to the sensing surface through the further bridging species; wherein the further bridging species comprises a product of a photo-initiated reaction between a fifth functional group connected to the sensing surface via an anchor species and a sixth functional group connected to the analyte capture species.
